(19) United States
(12) Patent Application Publication
(10) Pub. No.: US 2014/0073526 A1
(43) Pub. Date: Mar. 13, 2014

(54) IMMUNE FUNCTION BIOMARKERS

(71) Applicant: Nestec SA, Vevey (CH)

(72) Inventors: Rondo Paul Middleton, Creve Coeur, MO (US); Ziad S. Ramadan, Manchester, MO (US); Serge Andre Dominique Rezzi, Semsales (CH); Sebastiano Collino, Lausanne (FR); Francois-Pierre Martin, Vuisternens-devant-Romont (CH)

(73) Assignee: Nestec SA, Vevey (CH)

(21) Appl. No.: 14/021,495

(22) Filed: Sep. 9, 2013

Related U.S. Application Data
(60) Provisional application No. 61/698,973, filed on Sep. 10, 2012.

(51) Int. Cl. C12Q 1/68; G01N 33/68

(52) U.S. Cl. CPC C12Q 1/6883; G01N 33/6863
USPC 506/9; 506/16; 506/18

(57) ABSTRACT

The invention provides biomarkers associated with age related immune function and the use of the biomarkers to identify compositions useful for strengthening immune function in animals and to determine if an animal is responding to treatment targeted to strengthen the immune system.

### CROSS REFERENCE TO RELATED APPLICATIONS

This application claims priority to U.S. Provisional Application No. 61/698973 filed Sep. 10, 2012, the disclosure of which is incorporated herein by this reference.

### BACKGROUND OF THE INVENTION

1. Field of the Invention

The invention relates generally to the field of nutritional support of health and immunity in animals. In particular, the invention provides biomarkers associated with immune function, particularly biomarkers associated with age related changes in immune function, the use of the biomarkers to identify compositions useful for strengthening immune function in animals, and to determine if an animal is responding to treatment targeted to strengthen the immune system.

2. Description of Related Art

The gradual decline in immune system function that accompanies aging is known as immune senescence. This decline involves both an animal's capacity to respond to infections and the development of long term immunity. In addition to infectious diseases, an older animal is also more susceptible to other clinical conditions such as cancer, cardiovascular disease, neurological disorders and chronic inflammatory disorders. The identification of biomarkers associated with aging can be used to characterize an animal's immune system functionality. It can also be used to detect agents useful for strengthening immune system function and to monitor the effectiveness of treatment.

Biomarkers associated with immune function are known. However, the known biomarkers are mostly proinflammatory proteins or pathogen specific gene expression. US 2007/0150202 to Weigand et al. describe the use of c-reactive proteins and cytokines such as interleukin-6 (IL-6) to assess pro-inflammatory immune health of an individual, US 2004/0038201 to Nau et al. describe stimulus specific gene expression profiles to detect infection by a pathogen. US 2005/0002862 to Alters et al. describe biological markers for evaluating therapeutic treatment of inflammation and autoimmune disorders.

Despite the availability of the approaches summarized above, there remains a need for biomarkers associated with age related immune function and for methods to screen for agents that can strengthen immune function. The present invention satisfies this need.

### SUMMARY OF THE INVENTION

It is, therefore, an object of the present invention to provide a combination comprising a plurality of biomarkers associated with immune function that are differentially expressed in samples from old animals compared with samples from young animals.

It is a further object of the invention to provide methods for determining if a composition is effective in strengthening the immune function in an animal.

It is another object of the invention to provide methods for determining if an animal is responding to treatment with a composition suitable for strengthening immune function.

One or more of these other objects are achieved using novel collections of biomarkers associated with immune function that are differentially expressed in samples from old animals compared with samples from young animals.

Other and further objects, features, and advantages of the invention will be readily apparent to those skilled in the art.

### DETAILED DESCRIPTION OF THE INVENTION

### Definitions

As used throughout, ranges are used herein as shorthand, so as to avoid having to set out at length and describe each and every value within the range. Any appropriate value within the range can be selected, where appropriate, as the upper value, lower value, or the terminus of the range. It is understood that any and all whole or partial integers between any ranges or intervals set forth herein are included herein.

As used herein and in the appended claims, the singular form of a word includes the plural, and vice versa, unless the context clearly dictates otherwise. Thus, the references “a,” “an,” and “the” are generally inclusive of the plurals of the respective terms. For example, reference to “an animal”, “a method”, or “a substance” includes a plurality of such “animals”, “methods”, or “substances”. Similarly, the words “comprise”, “comprises”, and “comprising” are to be interpreted inclusively rather than exclusively.

The term “animal” means a human or other animal, including avian, bovine, canine, equine, feline, hicrine, murine, ovine, and porcine animals. When the term is used in the context of comparing test subjects, the animals that are compared are animals of the same species and possibly of the same race or breed. A “companion animal” is any domesticated animal, and includes, without limitation, cats, dogs, rabbits, guinea pigs, ferrets, hamsters, mice, gerbils, horses, cows, goats, sheep, donkeys, pigs, and the like. Preferably, the animal is a human or a companion animal such as a canine or feline.

The term “differential expression” or “differentially expressed” means increased or unregulated gene expression or means decreased or downregulated gene expression as detected by the absence, presence, or change in the amount of transcribed messenger RNA or translated protein in a sample, or means an increase or decrease in the amount of protein present in a sample.

The term “sample” means any animal tissue or fluid containing, e.g., polynucleotides, polypeptides, antibodies, metabolites, and the like, including cells and other tissue containing DNA and RNA. Examples include adipose, blood, cartilage, connective, epithelial, lymphoid, muscle, nervous, sputum, and the like. A sample may be solid or liquid and may be DNA, RNA, cDNA, bodily fluids such as blood or urine, cells, cell preparations or soluble fractions or media aliquots thereof, chromosomes, organelles, and the like.

“Young” refers generally to an individual in young adulthood, i.e., matured past puberty or adolescence, as would be defined by species, or by strain, breed or ethnic group within a species, in accordance with known parameters. Typically a young canine is less than five years of age.

“Aged” or “old,” as used herein, refers to an individual who is physically or chronologically within the last 30% of its average life expectancy, as determined by species, or by strain, breed or ethnic group within a species, in accordance with known parameters. Typically n old canine is greater than ten years.

“Middle-aged” refers generally to an individual that is in between young and old. Typically a middle-aged canine is five to ten years of age.

The methods and compositions and other advances disclosed here are not limited to particular methodology, protocols, and reagents described herein because, as the skilled artisan will appreciate, they may vary. Further, the terminology used herein is for the purpose of describing particular embodiments only, and is not intended to and does not limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the invention, the preferred compositions, methods, articles of manufacture, or other means or materials are described herein.

All patents, patent applications, publications, and other references cited or referred to herein are incorporated herein by reference to the extent allowed by controlling law. The discussion of those references is intended merely to summarize the assertions made therein. No admission is made that any such patents, patent applications, publications or references, or any portion thereof, is relevant, material, or prior art. The right to challenge the accuracy and pertinence of any assertion of such patents, patent applications, publications, and other references as relevant, material, or prior art is specifically reserved.

### The Invention

In one aspect, the invention provides a combination comprising a plurality of biomarkers associated with immune function that are differentially expressed in samples from old animals compared with samples from young animals.

In another aspect, the invention provides a combination comprising a plurality of biomarkers associated with immune function that are differentially expressed in samples from middle-aged animals corn pared with samples from young animals.

In another aspect, the invention provides a method for determining if a composition is effective in strengthening the immune function in an animal comprising: (a) obtaining a baseline sample from the animal prior to administration of the composition; (b) analyzing the baseline sample for one or more biomarkers associated with immune function; (c) administering the composition to the animal for a suitable amount of time; (d) obtaining a treatment sample from the animal after completion of the suitable amount of time; (e) analyzing the treatment sample for one or more biomarkers associated with immune function; and (f) determining if the composition is effective if one or more biomarkers present in the baseline sample is differentially expressed in the treatment sample.

In another aspect, the invention provides methods for determining if an animal is responding to treatment with a composition suitable for strengthening immune function comprising: (a) obtaining a baseline sample from the animal prior to administration of the composition; (b) analyzing the baseline sample for one or more biomarkers associated with immune function: (c) administering the composition to the animal for a suitable amount of time; (d) obtaining a treatment sample from the animal after completion of the suitable amount of time; (e) analyzing the treatment sample for one or more biomarkers associated with immune function; and (f) determining if the animal is responding to treatment if one or more biomarker present in the baseline sample is differentially expressed in the treatment sample.

The inventions are based upon the discovery of biomarkers in immune cells that were differentially expressed in samples from old and middle-age animals compared to samples from young animals. The markers identified can be used to monitor the effectiveness of therapies targeted at improving the animals' immune function.

The biomarkers of the present invention were identified using multiple technologies including leukocyte gene expression changes, changes in cytokines, chemokines and adipokine proteins and immune cell population changes. In various embodiments, the biomarkers associated with immune function include proteins and genes.

In some embodiments, the biomarker associated with immune function is one or more gene expression markers selected from E2F4, ADORA2A, RBMX, MVP, PEA15, UTP3, BST2, SORBS3, CD74, CD24, CCND3, PRKAG2, MED15, DNAJC8, CNDP2, CFD, IFNGR2, GABPA, TLR8, CAPG, GOT2, ZYX, MOV10, VDAC3, GNB2L1, NCF4, RPL7, SETD1B, NUDCD3, CD151, UIMC1, PADI4, TMEM55B, UPP1, GLTSCR2, MBOAT1, C22orf36, HSPB6, MSH2, ZNFX1, KDELR1, TMED10, SREBF1, IQGAP1, GPR177, HSPA6, TBCB, TRUB2, SUV39H1, GABARAP, PRKCSH, CD9, ZNF598, GPI, NUDC, TBC1D1, ADC, GAPDH, MED8, PSMC4, ATXN7L3, NCF1, GLIPR2, PEX19, MINPP1, PTPN23, PKM2, FLJ20160, FCGR1B, ADPGK, CIAPIN1, ARHGDIA, RPAP1, CCDC61, SYVN1, PADI4, DDOST, TREX1, PDCD11, TTC31, MAP7D1, MAPKSP1, HPX, DDOST, DERL2, TGFB1, PIM1, MAN2B1, USP3, RNH1, EIF4B, RHOG, SLC25A1, ACSS2, DOK2, NUMB, UCP2, VDAC3, LOC401875, ANXA11, PHKG2, GLB1, NARS, CLK3, AGBL5, PPP2R5C, XPNPEP1, TUBA4A, JARID1C, ARL4C, G6PC3, FES, USP5, and IREB2. In a preferred embodiment, the biomarker associated with immune function is one or more gene expression markers selected from E2F4, ADORA2A, RBMX, MVP, PEA15, UTP3, BST2, SORBS3, CD74, CD24, CCND3, PRKAG2, MED15, DNAJC8, CNDP2, CFD, IFNGR2, GABPA, TLR8, CAPG, GOT2, ZYX, MOV10, VDAC3, GNB2L1, NCF4, RPL7, SETD1B, NUDCD3, CD151, and UIMC1. In a more preferred embodiment, the biomarker associated with immune function is one or more gene expression marker selected from E2F4, ADORA2A, RBMX, MVP, PEA15, UTP3, BST2, SORBS3, CD74, and CD24.

In another embodiment, the biomarker associated with immune function is one or more proteins selected from granulocyte-macrophage colony-stimulating factor (GMCSF), chemokine (C-X-C motif) ligand 1 (CXCL1) (aka KC), adiponectin, and interleukin-18 (IL-18).

In one embodiment, the biomarkers associated with immune function that are differentially expressed in samples from old animals compared with samples from young animals are one or more proteins selected from granulocyte-macrophage colony-stimulating factor (GMCSF), adiponectin, and interleukin-18 (IL-18).

In another embodiment, the biomarkers associated with immune function that are differentially expressed in samples from middle-aged animals compared with samples from young animals are one or more proteins selected from granulocyte-macrophage colony-stimulating factor (GMCSF), chemokine (C-X-C motif) ligand 1 (CXCL1), adiponectin, and interleukin-18 (IL-18).

Any sample that is of biological origin may be useful in the present invention. Examples include, but are not limited to, blood (serum/plasma), cerebral spinal fluid (CSF), urine, stool breath, saliva, or biopsy of any tissue. In one embodiment, the sample is a blood sample. In another embodiment, the sample is a red blood sample. In yet another embodiment, the sample is a white blood sample.

In various embodiments, the animal is a human or companion animal. Preferably, the companion animal is a canine such as a dog.

The suitable amount of time for administering a composition suitable for strengthening immune function is any amount of a time required to achieve a strengthened immune function. In one embodiment, the suitable amount of time is at least 4 weeks, preferably at least 2 months, more preferably at least 6 months.

In some embodiments, the method for determining if a composition is effective in strengthening the immune function in an animal is determined if one or more biomarkers present in the baseline sample is differentially expressed in the treatment sample. In one embodiment, the determination is based on if two or more biomarkers present in the baseline sample are differentially expressed in the treatment sample. In another embodiment, the determination is based on if three or more biomarkers present in the baseline sample are differentially expressed in the treatment sample.

In some embodiments, the method for determining if a composition is effective in strengthening the immune function in an animal is determined if the amount of biomarker present in the baseline sample is greater compared to the amount present in the treatment sample, wherein the biomarker is one or more selected from the group consisting of E2F4, ADORA2A, RBMX, MVP, UTP3, SORBS3, CD74, CCND3, MED15, DNAJC8, CFD, VDAC3, GNB2L1, RPL7, PADI4, GLTSCR2, HSPB6, IQGAP1, PRKCSH, CD9, NUDC, MINPP1, PKM2, ARHGDIA, PADI4, DDOST, PIM1, VDAC3, and IREB2. In a preferred embodiment, the biomarker is one or more selected from the group consisting of E2F4, ADORA2A, RBMX, MVP, UTP3, SORBS3, CD74, CCND3, MED15, DNAJC8, CFD, VDAC3, GMB2L1, and RPL7. In a more preferred embodiment, the biomarker is one or more selected from the group consisting of E2F4, ADORA2A, RBMX, MVP, UTP3, SORBS3, and CD74.

In some embodiments, the method for determining if a composition is effective in strengthening the immune function in an animal is determined if the amount of biomarker present in the baseline sample is less than compared to the amount present in the treatment sample, wherein the biomarker is one or more selected from the group consisting of PEA15, BST2, CD24, PRKAG2, CNDP2, IFNGR2, GABPA, TLR8, CAPG, GOT2, ZYX, MOV10, NCF4, SETD1B, NUDCD3, CD151, UIMC1, TMEM55B, UPP1, MBOAT1, C22orf36, MSH2, ZNFX1, KDELR1, TMED10, SREBF1, GPR177, HSPA6, TBCB, TRUB2, SUV39H1, GABARAP, ZNF598, GPI, TBC1D1, ADC, GAPDH, MED8, PSMC4, ATXN7L3, NCF1, GLIPR2, PEX19, PTPN23, FLJ20160, FCGR1B, ADPGK, CIAPIN1, RPAP1, CCDC61, SYVN1, DDOST, TREX1, PDCD11, TTC31, MAP7D1, MAPKSP1, HPX, DERL2, TGFB1, MAN2B1, USP3, RNH1, EIF4B, RHOG, SLC25A1, ACSS2, DOK2, NUMB, UCP2, LOC401875, ANXA11, PHKG2, GLB1, NARS, CLK3, AGBL5, PPP2R5C, XPNPEP1, TUBA4A, JARID1C, ARL4C, G6PC3, FES, and USP5. In a preferred embodiment, the biomarker is one or more selected from the group consisting of PEA15, BST2, CD24, PRKAG2, CNDP2, IFNGR2, GABPA, TLR8, CAPG, GOT2, ZYX, MOV10, NCF4, SETD1B, NUDCD3, CD151, and UIMC1. In a more preferred embodiment, the biomarker is one or more selected from the group consisting of PEA15, BST2, and CD24.

In some embodiments, the method for determining if an animal is responding to treatment with a composition suitable for strengthening immune function is determined if one or more biomarker present in the baseline sample is differentially expressed in the treatment sample. In one embodiment, the determination is based on if two or more biomarkers present in the baseline sample are differentially expressed in the treatment sample. In another embodiment, the determination is based on if three or more biomarkers present in the baseline sample are differentially expressed in the treatment sample

In some embodiments, the method for determining if an animal is responding to treatment with a composition suitable for strengthening immune function is determined if the amount of biomarker present in the baseline sample is greater compared to the amount present in the treatment sample, wherein the biomarker is one or more selected from the group consisting of E2F4, ADORA2A, RBMX, MVP, UTP3, SORBS3, CD74, CCND3, MED15, DNAJC8, CFD, VDAC3, GNB2L1, RPL7, PADI4, GLTSCR2, HSPB6, IQGAP1, PRKCSH, CD9, NUDC, MINPP1, PKM2, ARHGDIA, PADI4, DDOST, PIM1, VDAC3, and IREB2. In a preferred embodiment, the biomarker is one or more selected from the group consisting of E2F4, ADORA2A, RBMX, MVP, UTP3, SORBS3, CD74, CCND3, MED15, DNAJC8, CFD, VDAC3, GNB2L1, and RPL7. In a more preferred embodiment, the biomarker is one or more selected from the group consisting of E2F4, ADORA2A, RBMX, MVP, UTP3, SORBS3, and CD74.

In some embodiments, the method for determining if an animal is responding to treatment with a composition suitable for strengthening immune function is determined if the amount of biomarker present in the baseline sample is less than compared to the amount present in the treatment sample, wherein the biomarker is one or more selected from the group consisting of PEA15, BST2, CD24, PRKAG2, CNDP2, IFNGR2, GABPA, TLR8, CAPG, GOT2, ZYX, MOV10, NCF4, SETD1B, NUDCD3, CD151, UIMC1, TMEM55B, UPP1, MBOAT1, C22orf36, MSH2, ZNFX1, KDELR1, TMED10, SREBF1, GPR177, HSPA6, TBCB, TRUB2, SUV39H1, GABARAP, ZNF598, GPI, TBC1D1, ADC, GAPDH, MED8, PSMC4, ATXN7L3, NCF1, GLIPR2, PEX19, PTPN23, FLJ20160, FCGR1B, ADPGK, CIAPIN1, RPAP1, CCDC61, SYVN1, DDOST, TREX1, PDCD11, TTC31, MAP7D1, MAPKSP1, HPX, DERL2, TGFB1, MAN2B1, USP3, RNH1, EIF4B, RHOG, SLC25A1, ACSS2, DOK2, NUMB, UCP2, LOC401875, ANXA11, PHKG2, GLB1, NARS, CLK3, AGBL5, PPP2R5C, XPNPEP1, TUBA4A, JARID1C, ARL4C, G6PC3, FES, and USP5. In a preferred embodiment, the biomarker is one or more selected from the group consisting of PEA15, BST2, CD24, PRKAG2, CNDP2, IFNGR2, GABPA, TLR8, CAPG, GOT2, ZYX, MOV10, NCF4, SETD1B, NUDCD3, CD151, and UIMC1. In a more preferred embodiment, the biomarker is one or more selected from the group consisting of PEA15, BST2, and CD24.

In various embodiments of the invention, changes in gene expression may be measured in one or both of two ways; (1) measuring transcription through detection of mRNA produced by a particular gene; and (2) measuring translation through detection of protein produced by a particular transcript.

Decreased or increased expression can be measured at the RNA level using any of the methods well known in the art for the quantitation of polynucleotides, such as, for example, PCR (including, without limitation, RT-PCR and qPCR), RNase protection, Northern blotting, microarray, macroarray, and other hybridization methods. The genes that are assayed or interrogated according to the invention are typically in the form of mRNA or reverse transcribed mRNA. The genes may be cloned and/or amplified. The cloning itself does not appear to bias the representation of genes within a population. However, it may be preferable to use polyA+ RNA as a source, as it can be used with fewer processing steps.

Decreased or increased expression can be measured at the protein level using any of the methods well known in the art for protein quantitation, such as, for example, western blotting, ELISA, mass spectrometry, etc.

### EXAMPLES

Various aspects of the invention can be further illustrated by the following examples. It will be understood that these examples are provided merely for purposes of illustration and do not limit the scope of the invention disclosed herein unless otherwise specifically indicated.

### Example 1

Thirty-six (36) animals were used for a canine trial. This consisted of an n=12 for each of 3 age groups. Canine (years); less than 5, 5-10 and greater than 10. Animals were all spayed or neutered. Any animal with an infection, disease, fever, recently immunized or has been given medication within 10 days was not used. Blood collections were drawn in same 5-day workweek on animals lasted overnight. 1.5-2 mL of blood in 3 mL ACD tubes and 2, 6-8 mL aliquots of blood in lithium-heparin tubes was collected for canines. A small aliquot from the lithium heparin tubes (prior to WBC/RNA isolation/plasma collection) was used for blood differential staining. The 1.5-2 mLs in the ACD tube were placed in a 4° C. refrigeration pack and shipped overnight or same day for flow cytometry analysis. All remaining samples were processed according to Ambion® RiboPure™-Blood (Life Technologies, Grand island, N.Y.) protocol except the plasma (separated from the WBC/red blood cells in the Ambient protocol) was stored at −80° C.

Cell Population Analysis. Peripheral blood smear/differential stain was performed by drawing up blood into a plain capillary tube and placing of small drop of blood on one end of a microscope slide. A second slide was used to by touching the blood drop at a 45 degree angle and pushing the blood across the first slide making a mono-layered feathered edge smear. Blood was allowed to dry completely and stained with Wright Stain. One drop of immersion oil was placed in the middle of the blood smear and viewed on an Olympus® BX51 microscope (Shinjuku, Japan) at 100× magnification. Percentage of monocytes, lymphocytes, bands, mature neutrophils, eosinophils and basophils were determined.

A resistant z-score rule was applied to the outlier detection algorithm.

$${z}_{i} = \frac{Xi - \overline{X}}{\overline{S}}$$

Where X̅ and S̅ are the median and MAD. An outlier is called |zᵢ|>4. Outliers were excluded from further statistical treatments.

A two-way ANOVA analysis was performed to evaluate the effects of the two factors: age (young, middle-age and old) and gender (M, F) as well as their interaction. P values for both factors and their interaction were computed. Means and standard error for each age group were also computed.

A pair-wise T-test was used to compare the difference between means of the three age groups. Multiple comparisons were adjusted, using Hommel's method to control family-wise error. Statistical analysis included natural log (ln) of canine flow cytometry lymphocyte, granulocyte and monocyte data.

Table 1 shows canine peripheral blood leukocyte populations as determined by peripheral blood smear/differential stain (ds, % of total) and flow cytometry (fc). SE represents standard error of the mean and in represents natural log.

Table 2 shows a two-way ANOVA analysis of age and gender on canine peripheral blood leukocyte populations as determined by peripheral blood smear/differential stain (ds, % of total) and flow cytometry (fc). P values for age, gender and their interaction are indicated as well as for the pair-wise T-test between age groups. Ln represents natural log.

TABLE 1

| | Young | Middle | Old |
|---|---|---|---|
| | Mean ± SE | Mean ± SE | Mean ± SE |
| Neutrophils (ds) | 59.42 ± 2.72 | 62.25 ± 3.76 | 59.67 ± 2.06 |
| Lymphocytes (ds) | 33.08 ± 2.64 | 27.5 ± 3.35 | 30.5 ± 1.9 |
| Monocytes (ds) | 3.25 ± 0.45^{A} | 5.25 ± 0.6^{B} | 4.75 ± 0.59^{AB} |
| Eosinophils (ds) | 3.5 ± 0.53 | 5.36 ± 0.69 | 5.18 ± 0.59 |
| CD4 (fc) | 59.34 ± 1.75 | 42.66 ± 1.87 | 29.04 ± 2.26 |
| CD8 (fc) | 21.44 ± 1.62 | 30.73 ± 3.19 | 43.72 ± 2.79 |
| CD4/CD8(fc) | 2.99 ± 0.27 | 1.6 ± 0.2 | 0.71 ± 0.08 |
| CD4 + CD8 (fc) | 80.78 ± 0.78 | 73.38 ± 1.73 | 72.76 ± 1.99 |
| CD5 (fc) | 80.4 ± 1.47 | 70.27 ± 1.67 | 80.24 ± 2.58 |
| B cells (fc) | 9.53 ± 0.81 | 8.02 ± 1.05 | 8.75 ± 1.06 |
| Lymphocytes (ln, fc) | 16.36 ± 0.12 | 15.12 ± 0.24 | 14.88 ± 0.19 |
| Granulocytes (ln, fc) | 16.52 ± 0.08 | 16.3 ± 0 | 15.29 ± 0.27 |
| Monocytes (ln, fc) | 14.8 ± 0.09 | 15.05 ± 0.18 | 14.73 ± 0.1 |

TABLE 2

| | P Age | P Sex | P Age-Sex | P Yng-Mid | P Yng-Old | P Mid-Old |
|---|---|---|---|---|---|---|
| Neutrophils (ds) | 0.7704 | 0.8515 | 0.8696 | 0.9523 | 0.9523 | 0.9523 |
| Lymphocytes (ds) | 0.3822 | 0.9593 | 0.8052 | 0.4561 | 0.5022 | 0.5022 |
| Monocytes (ds) | 0.0487 | 0.4525 | 0.7747 | 0.0456 | 0.1269 | 0.5264 |
| Eosinophils (ds) | 0.1124 | 0.5769 | 0.4196 | 0.1169 | 0.1559 | 0.841 |
| CD4 (fc) | 0 | 0.5139 | 0.0139 | 0 | 0 | 0 |
| CD8 (fc) | 0 | 0.2284 | 0.0599 | 0.0173 | 0 | 0.0026 |
| CD4/CD8 (fc) | 0 | 0.1249 | 0.1463 | 0 | 0 | 0.0037 |
| CD4 + CD8 (fc) | 0.002 | 0.2638 | 0.6745 | 0.0047 | 0.0033 | 0.7825 |
| CD5 (fc) | 6.00E−04 | 0.3784 | 0.0686 | 0.0018 | 0.955 | 0.0022 |
| B cells (fc) | 0.5295 | 0.0508 | 0.4084 | 0.6013 | 0.6013 | 0.6013 |
| Lymphocytes (ln, fc) | 0 | 0.3794 | 0.0042 | 1.00E−04 | 0 | 0.3797 |
| Granulocytes (ln, fc) | 0 | 0.8159 | 0.6731 | 0.3264 | 0 | 2.00E−04 |
| Monocytes (ln, fc) | 0.1626 | 0.5488 | 0.0224 | 0.3761 | 0.6984 | 0.2821 |

### Example 2

Microarray construction. Lymphocytes were isolated from whole blood and total RNA was extracted. Lymphocytes were also isolated and cultured. These were stimulated with various immunological agents (see Table 1 and Table 2 for identity, level and duration). After stimulation, total RNA was extracted and combined with the above RNA. RNA was checked for quality and quantity and shipped to Invitrogen for construction of normalized cDNA libraries. The pCMVSport 6.1 vector was used for cloning in DH10B-Ton A bacteria. Normalization resulted in an 80-fold reduction in beta-actin message with a 96% vector insert rate.

The libraries were plated and approximately 2550 colonies were isolated. Once these were amplified by growth, the associated vectors were isolated and sequenced. Sequencing quality was assessed using phred scores of >=20. (phred scores are defined as −log(1 error/number of bases) there for a phred score of 20 is defined as one or fewer errors per 100 bases) This resulted in 92% good quality sequences. cDNA vector inserts were amplified by PCR in 27, 96-well plates. They were then spotted onto prepared microarray slides. The resulting microarrays now represent medium-density lymphocyte gene microarrays of approximately 5100 spots containing 2550 gene targets in duplicate.

Microarray analysis: cDNA was synthesized from 6 ug total RNA according to manufacturer's directions (Genisphere, kit H500130). Briefly, primers constructed with an extension sequence to capture a Cy3 label were incubated with RNA at 80° C. for 10 minutes. Superscript™ II ((Life Technologies, Grand Island, N.Y.) reverse transcriptase was used according to manufacturer's directions. Reverse transcription was performed at 42° C. for 2 hours. Reaction was stopped by the addition of NaOH/EDTA, incubated at 65° C. for 10 minutes and Tris-HCL, pH 7.5 was added to neutralize, cDNA was isolated using Microcon® YM-30 (EMD Millipore Corp., Billerica, Mass.) columns according to manufacturer's directions. Microarray hybridization, washes and slide drying procedures were carried out in an automated Tecan HS 4800™ hybridization system (Tecan Croup Ltd., Männedorf, Switzerland). Briefly, microarrays were hybridized at 38° C. for 18 hours They were washed with 2× SSC, 0.2% SDS (20× SSC; 175.3 g Sodium Chloride and 88.2 g Sodium Citrate per liter, pH 7, 10% SDS; 100 g Sodium Lauryi. Sulfate per liter, pH 7.2) @ 42° C., 2× SSC @ 23° C. and 0.2× SSC @ 23° C. The Cy3 label was added to the microarrays and hybed at 23° C. for 3 hours. The previous wash steps were repeated. The microarrays were dried using a Nitrogen gas purge for 2 miutes-30 seconds.

Transcriptomics. 19 canines from the old and young group were used to investigate leukocyte gene expression changes. After 2 were removed due to poor correlation a total of 17 were used with 10 coming from the young group (<5 years of age) and 7 coming from the old group (>10 years of age).

Gene ID, signal median, background median, and quality control flag information were extracted from the raw data. A gene's expression was determined as the difference between its signal median and its background median. Genes with gene ID as “BLANK”, “Alien”, “n/a”, “blank” or “Blank” were removed. Quality control flagged genes were also eliminated. Within an array, two technical duplicates were combined and their average was calculated. Binary logarithm transformation was used for each gene's expression.

Including the omission of quality controlled flagged spots from the microarray analysis, there was approximately 50% missing data (considering the entire probe-set on the microarray) for the canine analysis. Non-linear cubic spline normalization method was used.

A two-way ANOVA analysis was performed to evaluate the effects of the two factors: age (young, old, see Results) and gender (M, F) as well as their interaction. P values for both factors and their interaction were computed. A T-test was used to compare the difference between means of die two age groups. P values and means of each age group were computed. Each age group should have at least two valid data points in order to enter the comparison with other groups. Canine leukocyte age-related transcriptional changes (p<0.05) are shown in Table 3.

TABLE 3

| | | Mean | Mean | Fold | Gene | |
|---|---|---|---|---|---|---|
| Probe ID | P-Value | Young | Old | Charge | Symbol | Description |
| CR6F9 | 0.001 | 7.23 | 8.27 | 2.07 | NCF4 | Homo sapiens neutrophil cytosolic factor 4, |
| | | | | | | 40 kDa (NCF4), transcript variant 1, mRNA |
| CR16E7 | 0.001 | 5.38 | 6.20 | 1.77 | TRUB2 | Homo sapiens TruB pseudouridine (psi) |
| | | | | | | synthase homolog 2 (E. coli) (TRUB2), |
| | | | | | | mRNA |
| CR7F10 | 0.001 | 5.75 | 6.46 | 1.63 | MED8 | Homo sapiens mediator complex subunit 8 |
| | | | | | | (MED8), transcript transcript variant |
| CR18B7 | 0.001 | 5.32 | 6.17 | 1.81 | NA | NA |
| CR5E7 | 0.001 | 5.12 | 5.59 | 1.38 | LOC401875 | PREDICTED: Homo sapiens misc_RNA |
| | | | | | | (LOC401875), (LOC401875), miscRNA. |
| CR27F8 | 0.002 | 4.87 | 5.89 | 2.03 | SETD1B | Homo sapiens SET domain containing 1B |
| | | | | | | (SETD1B), mRNA |
| CR27F3 | 0.002 | 5.56 | 5.98 | 1.34 | AGBL5 | Homo sapiens ATP/GTP binding protein- |
| | | | | | | like 5 (AGBL5), (AGBL5), transcript |
| CR18D11 | 0.002 | 8.43 | 6.66 | −3.43 | UTP3 | Homo sapiens UTP3, small subunit (SSU) |
| | | | | | | processome component, homolog (S. |
| | | | | | | cerevisiae) (UTP3), mRNA |
| CR11C4 | 0.003 | 6.48 | 7.07 | 1.51 | TREX1 | Homo sapiens three prime repair |
| | | | | | | exonuclease 1 (TREX1), (TREX1), |
| | | | | | | transcript |
| CR17B4 | 0.003 | 4.58 | 5.84 | 2.40 | NA | NA |
| CR13E5 | 0.003 | 5.94 | 3.80 | −4.42 | MVP | Homo sapiens major vault protein (MVP), |
| | | | | | | transcript variant 2, 2, mRNA. |
| CR15D12 | 0.004 | 5.67 | 6.24 | 1.49 | MAPKSP1 | Homo sapiens MAPK scaffold protein 1 |
| | | | | | | (MAPKSP1), transcript variant |
| CR20E2 | 0.005 | 1.88 | 3.92 | 4.12 | PEA15 | Homo sapiens phosphoprotein enriched in |
| | | | | | | astrocytes 15 (PEA15), |
| CR17G7 | 0.005 | 7.10 | 5.62 | −2.78 | MED15 | Homo sapiens mediator complex subunit 15 |
| | | | | | | (MED15), transcript |
| CR24E7 | 0.005 | 4.43 | 5.23 | 1.75 | NA | NA |
| CR23H12 | 0.005 | 5.54 | 6.30 | 1.69 | GPI | Homo sapiens glucose phosphate isomerase |
| | | | | | | (GPI), (GPI), mRNA. |
| CR2E10 | 0.005 | 3.95 | 4.81 | 1.81 | HSPA6 | Homo sapiens heat shock 70 kDa protein 6 |
| | | | | | | (HSP70B′) (HSPA6), (HSPA6), mRNA. |
| CR10D4 | 0.005 | 5.93 | 6.53 | 1.51 | DDOST | Homo sapiens sapiens dolichyl- |
| | | | | | | diphosphooligosaccharide-protein |
| CR4G6 | 0.006 | 5.16 | 5.60 | 1.36 | NA | NA |
| CR17C12 | 0.006 | 4.74 | 5.37 | 1.55 | NA | NA |
| CR18D5 | 0.006 | 4.19 | 5.09 | 1.86 | SREBF1 | Homo sapiens sterol regulatory element |
| | | | | | | binding transcription factor 1 (SREBF1), |
| | | | | | | transcript variant 2, mRNA |
| CR17E8 | 0.006 | 6.30 | 7.39 | 2.12 | NA | NA |
| CR17D11 | 0.007 | 4.86 | 5.50 | 1.56 | NA | NA |
| CR10A12 | 0.007 | 3.77 | 5.26 | 2.81 | PRKAG2 | Homo sapiens protein kinase, AMP- |
| | | | | | | activated, gamma 2 non-catalytic subunit |
| | | | | | | (PRKAG2), transcript variant a, mRNA |
| CR13C2 | 0.007 | 6.38 | 5.40 | −1.98 | PADI4 | Homo sapiens peptidyl arginine deiminase, |
| | | | | | | type IV (PADI4), (PADI4), mRNA. |
| CR8E4 | 0.007 | 7.31 | 8.27 | 1.95 | UPP1 | Homo sapiens uridine phosphorylase 1 |
| | | | | | | (UPP1), transcript variant 1 |
| CR17H10 | 0.008 | 8.08 | 7.31 | −1.71 | CD9 | Homo sapiens CD9 molecule (CD9), |
| | | | | | | (CD9), mRNA. |
| CR17G5 | 0.008 | 5.67 | 6.11 | 1.35 | PHKG2 | Homo sapiens phosphorylase kinase, |
| | | | | | | gamma 2 (testis) (PHKG2), (PHKG2), |
| | | | | | | mRNA. |
| CR8H12 | 0.008 | 5.07 | 5.97 | 1.87 | TMED10 | Homo sapiens transmembrane emp24-like |
| | | | | | | trafficking protein 10 (yeast) (TMED10), |
| | | | | | | mRNA |
| CR14H9 | 0.008 | 7.39 | 7.99 | 1.51 | NA | NA |
| CR9B9 | 0.009 | 6.46 | 6.87 | 1.32 | PPP2R5C | Homo sapiens protein phosphatase 2, |
| | | | | | | regulatory subunit B′, B′, gamma |
| CR7F11 | 0.009 | 5.95 | 6.59 | 1.56 | NA | NA |
| CR25E2 | 0.009 | 5.52 | 6.54 | 2.02 | CD151 | Homo sapiens CD151 molecule (Raph |
| | | | | | | blood group) (CD151), transcript |
| CR24A3 | 0.009 | 6.05 | 6.66 | 1.53 | CCDC61 | Homo sapiens coiled-coil domain |
| | | | | | | containing 61 (CCDC61), (CCDC61), |
| | | | | | | mRNA. |
| CR13G10 | 0.009 | 6.80 | 7.99 | 2.29 | CAPG | Homo sapiens capping protein (actin |
| | | | | | | filament), filament), gelsolin-like |
| CR8E8 | 0.009 | 5.14 | 5.90 | 1.69 | NA | NA |
| CR27A8 | 0.010 | 5.30 | 5.95 | 1.57 | FLJ20160 | Homo sapiens FLJ20160 protein |
| | | | | | | (FLJ20160), (FLJ20160), mRNA. |
| CR15F10 | 0.010 | 7.79 | 9.38 | 3.00 | CD24 | Homo sapiens CD24 molecule (CD24), |
| | | | | | | mRNA |
| CR11E11 | 0.010 | 6.26 | 6.60 | 1.27 | ARL4C | Homo sapiens ADP-ribosylation factor-like |
| | | | | | | 4C (ARL4C), mRNA |
| CR9F9 | 0.010 | 8.13 | 8.93 | 1.74 | GABARAP | Homo sapiens GABA(A) receptor- |
| | | | | | | associated protein (GABARAP), mRNA |
| CR11A2 | 0.010 | 5.11 | 5.59 | 1.40 | ACSS2 | Homo sapiens acyl-CoA synthetase short- |
| | | | | | | chain family member member 2 |
| CR2SC2 | 0.010 | 4.73 | 5.41 | 1.60 | PEX19 | Homo sapiens peroxisomal biogenesis |
| | | | | | | factor 19 (PEX19), transcript variant 1, |
| | | | | | | mRNA |
| CR17A6 | 0.011 | 5.73 | 6.02 | 1.22 | G6PC3 | Homo sapiens, glucose 6 phosphatase, |
| | | | | | | catalytic, 3 (G6PC3), transcript variant 1, |
| | | | | | | mRNA |
| CR7B10 | 0.011 | 8.94 | 7.84 | −2.15 | GNB2L1 | Homo sapiens guanine nucleotide binding |
| | | | | | | protein (G protein), beta polypeptide 2-like |
| | | | | | | 1 (GNB2L1), mRNA |
| CR8F1 | 0.011 | 6.52 | 4.93 | −3.01 | CD74 | Homo sapiens CD74 molecule, major |
| | | | | | | histocompatibility complex, class |
| CR18G5 | 0.011 | 3.29 | 4.67 | 2.60 | NA | NA |
| CR2G8 | 0.012 | 5.70 | 6.62 | 1.89 | KDELR1 | Homo sapiens KDEL (Lys-Asp-Glu-Leu) |
| | | | | | | endoplasmic reticulum reticulum protein |
| CR27H9 | 0.012 | 6.37 | 7.61 | 2.37 | IFNGR2 | Homo sapiens interferon gamma receptor 2 |
| | | | | | | (interferon gamma transducer 1) (IFNGR2), |
| | | | | | | mRNA |
| CR14D8 | 0.012 | 5.13 | 5.76 | 1.55 | CIAPIN1 | Homo sapiens cytokine induced apoptosis |
| | | | | | | inhibitor 1 1 (CIAPIN1), |
| CR5A8 | 0.012 | 6.53 | 7.04 | 1.43 | MAN2B1 | Homo sapiens mannosidase, alpha, class |
| | | | | | | 2B, member 1 (MAN2B1), (MAN2B1), |
| | | | | | | mRNA. |
| CR20C11 | 0.012 | 6.63 | 7.80 | 2.25 | GOT2 | Homo sapiens glutamic-oxaloacetic |
| | | | | | | transaminase 2, mitochondrial (aspartate |
| | | | | | | aminotransferase 2) (GOT2), nuclear gene |
| | | | | | | encoding mitochondrial protein, mRNA |
| CR15C2 | 0.012 | 4.88 | 5.57 | 1.62 | NCF1 | Homo sapiens neutrophil cytosolic factor 1 |
| | | | | | | (NCF1), (NCF1), mRNA. |
| CR11C3 | 0.012 | 5.85 | 6.38 | 1.44 | NA | NA |
| CR19E7 | 0.013 | 5.24 | 6.09 | 1.81 | NA | NA |
| CR16C8 | 0.013 | 2.75 | 4.10 | 2.54 | CNDP2 | Homo sapiens CNDP dipeptidase 2 |
| | | | | | | (metallopeptidase M20 family) (CNDP2), |
| | | | | | | mRNA |
| CR10G3 | 0.013 | 4.97 | 5.40 | 1.35 | NA | NA |
| CR23G4 | 0.014 | 4.10 | 3.54 | −1.47 | DDOST | Homo sapiens sapiens dolichyl- |
| | | | | | | diphosphooligosaccharide-protein |
| CR1H1 | 0.014 | 5.81 | 5.29 | −1.43 | PIM1 | Homo sapiens pim-1 oncogene (PIM1), |
| | | | | | | (PIM1), mRNA. |
| CR24D12 | 0.014 | 5.64 | 6.78 | 2.21 | MOV10 | Homo sapiens Mov10, Moloney leukemia |
| | | | | | | virus 10, homolog homolog (mouse) |
| CR3H8 | 0.014 | 6.41 | 3.67 | −6.67 | RBMX | Homo sapiens RNA binding motif protein, |
| | | | | | | X-linked (RBMX), (RBMX), mRNA. |
| CR7H12 | 0.015 | 5.76 | 6.34 | 1.49 | MAP7D1 | Homo sapiens MAP7 domain containing 1 |
| | | | | | | (MAP7D1), (MAP7D1), mRNA. |
| CR17E11 | 0.016 | 4.84 | 5.64 | 1.74 | SUV39H1 | Homo sapiens suppressor of variegation 3-9 |
| | | | | | | homolog 1 1 (Drosophila) |
| CR25F7 | 0.016 | 4.19 | 5.12 | 1.90 | ZNFX1 | Homo sapiens zinc finger, NFX1-type |
| | | | | | | containing 1 (ZNFX1), mRNA |
| CR13H4 | 0.016 | 11.98 | 12.74 | 1.70 | ZNF598 | Homo sapiens zinc finger protein 598 |
| | | | | | | (ZNF598), (ZNF598), mRNA. |
| CR21B9 | 0.016 | 3.10 | 4.80 | 3.26 | BST2 | Homo sapiens bone marrow stromal cell |
| | | | | | | antigen 2 (BST2), mRNA |
| CR17C6 | 0.017 | 4.71 | 5.40 | 1.62 | ATXN7L3 | Homo sapiens ataxin 7-like 3 (ATXN7L3), |
| | | | | | | transcript variant 2, 2, mRNA. |
| CR5C9 | 0.017 | 6.71 | 7.17 | 1.38 | ANXA11 | Homo sapiens annexin A11 (ANXA11), |
| | | | | | | transcript variant a, a, mRNA. |
| CR27G11 | 0.017 | 9.27 | 7.72 | −2.93 | NA | NA |
| CR14F7 | 0.018 | 5.49 | 6.12 | 1.55 | ADPGK | Homo sapiens ADP-dependent glucokinase |
| | | | | | | (ADPGK), transcript transcript variant |
| CR11E4 | 0.018 | 6.79 | 7.28 | 1.41 | RHOG | Homo sapiens ras homolog gene family, |
| | | | | | | member G (rho G) (RHOG), mRNA |
| CR22F10 | 0.018 | 4.33 | 5.17 | 1.79 | TBCB | Homo sapiens tubulin folding cofactor B |
| | | | | | | (TBCB), (TBCB), mRNA. |
| CR18H3 | 0.018 | 6.48 | 7.49 | 2.02 | NA | NA |
| CR13A4 | 0.020 | 4.79 | 5.77 | 1.97 | TMEM55B | Homo sapiens transmembrane protein 55B |
| | | | | | | (TMEM55B), transcript variant 2, mRNA |
| CR10G1 | 0.020 | 8.10 | 7.43 | −1.59 | NA | NA |
| CR10A9 | 0.021 | 5.15 | 5.58 | 1.35 | GLB1 | Homo sapiens galactosidase, beta 1 |
| | | | | | | (GLB1), transcript variant variant 1. |
| CR22F3 | 0.021 | 4.96 | 4.21 | −1.68 | NUDC | Homo sapiens nuclear distribution gene C |
| | | | | | | homolog (A. (A. nidulans) |
| CR12E8 | 0.021 | 5.76 | 6.33 | 1.48 | NA | NA |
| CR11A5 | 0.021 | 6.42 | 7.00 | 1.50 | PDCD11 | Homo sapiens programmed cell death 11 |
| | | | | | | (PDCD11), (PDCD11), mRNA. |
| CR1B11 | 0.021 | 5.38 | 5.87 | 1.40 | DOK2 | Homo sapiens docking protein 2, 56 kDa |
| | | | | | | (DOK2), (DOK2), mRNA. |
| CR24C5 | 0.021 | 5.62 | 6.16 | 1.45 | TGFB1 | Homo sapiens transforming growth factor, |
| | | | | | | beta 1 (TGFB1), (TGFB1), mRNA. |
| CR7E2 | 0.022 | 4.65 | 5.81 | 2.23 | ZYX | Homo sapiens zyxin (ZYX), transcript |
| | | | | | | variant 1, mRNA |
| CR14H12 | 0.022 | 5.28 | 5.90 | 1.54 | NA | NA |
| CR14H11 | 0.022 | 5.75 | 6.26 | 1.43 | USP3 | Homo sapiens ubiquitin specific peptidase 3 |
| | | | | | | (USP3), (USP3), mRNA. |
| CR12B11 | 0.023 | 8.71 | 7.91 | −1.74 | PRKCSH | Homo sapiens protein kinase C substrate |
| | | | | | | 80K-H (PRKCSH), transcript variant 2, |
| | | | | | | mRNA |
| CR14G10 | 0.024 | 4.75 | 5.62 | 1.83 | GPR177 | Homo sapiens G protein-coupled receptor |
| | | | | | | 177 (GPR177), (GPR177), transcript |
| CR12F12 | 0.024 | 4.98 | 3.29 | −3.22 | SORBS3 | Homo sapiens sorbin and SH3 domain |
| | | | | | | containing 3 3 (SORBS3). |
| CR19C8 | 0.024 | 4.43 | 0.64 | −13.75 | E2F4 | Homo sapiens E2F transcription factor 4, |
| | | | | | | p107/p130-binding (E2F4), mRNA |
| CR6G12 | 0.024 | 7.22 | 8.23 | 2.02 | UIMC1 | Homo sapiens ubiquitin interaction motif |
| | | | | | | containing 1 1 (UIMC1), |
| CR4H9 | 0.024 | 6.70 | 6.97 | 1.21 | USP5 | Homo sapiens ubiquitin specific peptidase 5 |
| | | | | | | (isopeptidase T) (USP5), transcript variant |
| | | | | | | 2, mRNA |
| CR2B11 | 0.025 | 7.06 | 8.08 | 2.03 | NUDCD3 | Homo sapiens NudC domain containing 3 |
| | | | | | | (NUDCD3), mRNA |
| CR27E1 | 0.025 | 7.90 | 8.92 | 2.02 | NA | NA |
| CR11D9 | 0.025 | 6.53 | 7.01 | 1.40 | NUMB | Homo sapiens numb homolog (Drosophila) |
| | | | | | | (NUMB), transcript transcript variant |
| CR24B3 | 0.026 | 5.49 | 5.33 | −1.12 | IREB2 | Homo sapiens iron-responsive element |
| | | | | | | binding protein 2 (IREB2), mRNA |
| CR11G12 | 0.027 | 5.86 | 6.59 | 1.66 | ADC | Homo sapiens arginine decarboxylase |
| | | | | | | (ADC), (ADC), mRNA. |
| CR16F12 | 0.027 | 8.38 | 7.08 | −2.45 | CFD | Homo sapiens complement factor D |
| | | | | | | (adipsin) (CFD), mRNA |
| CR9E6 | 0.028 | 6.23 | 6.62 | 1.31 | XPNPEP1 | Homo sapiens X-prolyl aminopeptidase |
| | | | | | | (aminopeptidase P) 1, 1, soluble |
| CR13H5 | 0.028 | 4.28 | 0.60 | −12.82 | ADORA2A | Homo sapiens adenosine A2a receptor |
| | | | | | | (ADORA2A), mRNA |
| CR11B7 | 0.029 | 11.60 | 12.32 | 1.64 | GAPDH | Homo sapiens glyceraldehyde-3-phosphate |
| | | | | | | dehydrogenase (GAPDH), mRNA |
| CR26F11 | 0.030 | 5.43 | 5.97 | 1.45 | NA | NA |
| CR1B7 | 0.030 | 6.33 | 5.68 | −1.57 | PKM2 | Homo sapiens pyruvate kinase, muscle |
| | | | | | | (PKM2), transcript variant variant 1. |
| CR4E2 | 0.030 | 4.24 | 3.28 | −1.95 | GLTSCR2 | Homo sapiens glioma tumor suppressor |
| | | | | | | candidate region gene gene 2 |
| CR11C7 | 0.031 | 6.31 | 6.64 | 1.35 | NARS | Homo sapiens asparaginyl-tRNA synthetase |
| | | | | | | (NARS), (NARS), mRNA. |
| CR22E4 | 0.031 | 3.84 | 4.80 | 1.94 | MBOAT1 | Homo sapiens membrane bound O- |
| | | | | | | acyltransferase domain containing |
| | | | | | | containing 1 |
| CR17C3 | 0.032 | 4.87 | 5.48 | 1.53 | RPAP1 | Homo sapiens RNA polymerase II |
| | | | | | | associated protein 1 (RPAP1), (RPAP1), |
| | | | | | | mRNA. |
| CR9H1 | 0.032 | 5.14 | 5.75 | 1.53 | SYVN1 | Homo sapiens, synovial apoptosis inhibitor |
| | | | | | | 1, synoviolin (SYVN1), transcript variant 1, |
| | | | | | | mRNA |
| CR11G2 | 0.032 | 5.95 | 6.30 | 1.28 | JARID1C | Homo sapiens jumonji, AT rich interactive |
| | | | | | | domain 1C 1C (JARID1C), |
| CR22D11 | 0.033 | 4.16 | 2.74 | −2.68 | DNAJC8 | Homo sapiens DnaJ (Hsp40) homolog, |
| | | | | | | subfamily C, member 8 8 (DNAJC8), |
| CR13A10 | 0.033 | 5.47 | 5.95 | 1.40 | NA | NA |
| CR9D12 | 0.034 | 4.89 | 3.14 | −3.38 | NA | NA |
| CR26A5 | 0.034 | 7.45 | 8.10 | 1.58 | PTPN23 | Homo sapiens protein tyrosine phosphatase, |
| | | | | | | non-receptor type 23 (PTPN23), mRNA |
| CR9B2 | 0.035 | 9.83 | 8.79 | −2.06 | RPL7 | Homo sapiens ribosomal protein L7 |
| | | | | | | (RPL7), mRNA |
| CR25H8 | 0.035 | 3.62 | 4.84 | 2.33 | GABPA | Homo sapiens GA binding protein |
| | | | | | | transcription factor, alpha subunit 60 kDa |
| | | | | | | (GABPA), mRNA |
| CR15A2 | 0.035 | 5.39 | 4.44 | −1.93 | HSPB6 | Homo sapiens heat shock protein, alpha- |
| | | | | | | crystallin-related, B6 (HBPB6), mRNA |
| CR6C5 | 0.036 | 6.22 | 6.87 | 1.57 | FCGR1B | Homo sapiens Fc fragment of IgG, high |
| | | | | | | affinity 1b, receptor receptor (CD64) |
| CR27E3 | 0.036 | 4.75 | 5.51 | 1.70 | NA | NA |
| CR25F8 | 0.036 | 6.61 | 5.96 | −1.58 | MINPP1 | Homo sapiens multiple inositol |
| | | | | | | polyphosphate histidine phosphatase, 1 |
| | | | | | | (MINPP1), mRNA |
| CR25A12 | 0.036 | 6.90 | 7.86 | 1.94 | C22orf36 | Homo sapiens chromosome 22 open |
| | | | | | | reading frame 36 (C22orf36), |
| CR13A11 | 0.036 | 3.59 | 4.80 | 2.31 | TLR8 | Homo sapiens toll-like receptor 8 (TLR8), |
| | | | | | | (TLR8), mRNA. |
| CR17H2 | 0.037 | 5.72 | 6.30 | 1.50 | TTC31 | Homo sapiens tetratricopeptide repeat |
| | | | | | | domain 31 (TTC31), transcript variant 1, |
| | | | | | | mRNA |
| CR14E6 | 0.037 | 6.01 | 6.29 | 1.22 | FES | Homo sapiens feline sarcoma oncogene |
| | | | | | | (FES), (FES), mRNA. |
| CR3H5 | 0.038 | 5.92 | 5.30 | −1.54 | ARHGDIA | Homo sapiens Rho GDP dissociation |
| | | | | | | inhibitor (GDI) alpha (ARHGDIA), mRNA |
| CR24E6 | 0.039 | 5.29 | 5.85 | 1.48 | HPX | Homo sapiens hemopexin (HPX), mRNA |
| CR12D1 | 0.039 | 4.82 | 5.37 | 1.47 | DERL2 | Homo sapiens Der1-like domain family, |
| | | | | | | member 2 (DERL2), (DERL2), mRNA. |
| CR17A8 | 0.039 | 5.19 | 5.71 | 1.43 | RNH1 | Homo sapiens ribonuclease/angiogenin |
| | | | | | | inhibitor 1 (RNH1), transcript variant 1, |
| | | | | | | mRNA |
| CR12D7 | 0.039 | 4.15 | 4.93 | 1.71 | NA | NA |
| CR17F12 | 0.040 | 5.09 | 5.58 | 1.41 | SLC25A1 | Homo sapiens solute carrier family 25 |
| | | | | | | (mitochondrial (mitochondrial carrier: |
| CR6F2 | 0.040 | 5.86 | 6.23 | 1.30 | NA | NA |
| CR11H9 | 0.040 | 7.56 | 8.05 | 1.41 | EIF4B | Homo sapiens eukaryotic translation |
| | | | | | | initiation factor 4B (EIF4B), mRNA |
| CR23H4 | 0.040 | 3.93 | 4.71 | 1.71 | NA | NA |
| CR7H11 | 0.040 | 7.47 | 8.16 | 1.61 | GLIPR2 | Homo sapiens GL1 pathogenesis-related 2 |
| | | | | | | (GLIPR2), (GLIPR2), mRNA. |
| CR17B11 | 0.040 | 7.18 | 6.72 | −1.38 | VDAC3 | Homo sapiens voltage-dependent anion |
| | | | | | | channel 3 (VDAC3), (VDAC3), mRNA. |
| CR25A4 | 0.042 | 4.23 | 4.97 | 1.68 | TBC1D1 | Homo sapiens TBC1 (tre-2/USP6, BUB2, |
| | | | | | | cdc16) domain family, member 1 |
| | | | | | | (TBC1D1), mRNA |
| CR14A12 | 0.042 | 7.71 | 6.60 | −2.16 | VDAC3 | Homo sapiens voltage-dependent anion |
| | | | | | | channel 3 (VDAC3), (VDAC3), mRNA. |
| CR9E12 | 0.042 | 3.88 | 5.22 | 2.52 | NA | NA |
| CR18E3 | 0.042 | 4.99 | 5.69 | 1.62 | PSMC4 | Homo sapiens proteasome (prosome, |
| | | | | | | macropain) 26S subunit, ATPase, ATPase, 4 |
| CR24A1 | 0.042 | 3.82 | 4.77 | 1.94 | NA | NA |
| CR25D4 | 0.043 | 7.64 | 7.04 | −1.52 | PADI4 | Homo sapiens peptidyl arginine deiminase, |
| | | | | | | type IV (PADI4), (PADI4), mRNA. |
| CR22A3 | 0.043 | 5.54 | 6.58 | 2.06 | NA | NA |
| CR2F7 | 0.043 | 5.65 | 6.07 | 1.34 | CLK3 | Homo sapiens CDC-like kinase 3 (CLK3), |
| | | | | | | transcript variant 1, 1, mRNA. |
| CR18D7 | 0.043 | 5.82 | 6.33 | 1.43 | NA | NA |
| CR27G9 | 0.044 | 6.10 | 6.58 | 1.40 | UCP2 | Homo sapiens uncoupling protein 2 |
| | | | | | | (mitochondrial, proton carrier) (UCP2), |
| | | | | | | nuclear gene encoding mitochondrial |
| | | | | | | protein, mRNA |
| CR20G5 | 0.046 | 5.40 | 4.51 | −1.85 | IQGAP1 | Homo sapiens IQ motif containing GTPase |
| | | | | | | activating protein protein 1 |
| CR14G4 | 0.046 | 5.43 | 6.38 | 1.93 | NA | NA |
| CR1D3 | 0.047 | 4.94 | 3.42 | −2.86 | CCND3 | Homo sapiens cyclin D3 (CCND3), |
| | | | | | | (CCND3), mRNA. |
| CR12B7 | 0.048 | 3.81 | 4.75 | 1.91 | MSH2 | Homo sapiens mutS homolog 2, colon |
| | | | | | | cancer, nonpolyposis type 1 1 (E. |
| CR4G3 | 0.048 | 5.16 | 5.48 | 1.26 | NA | NA |
| CR8B5 | 0.049 | 7.17 | 7.53 | 1.28 | TUBA4A | Homo sapiens tubulin, alpha 4a (TUBA4A), |
| | | | | | | mRNA |

### Example 3

Protein Analysis. Cytokine/chemokine/adipokine Analysis. Cytokine, chemokine and adipokine protein levels were determined using the LINCOplex™ Kit according to manufacturer's directions (Linco Research, Inc., St. Charles, Mo.). Specifically, 200 ul of Wash buffer was added per well and shaken 10 min at room temp. This was vacuumed out and 25 ul standards, controls and background (assay buffer) was added to appropriate wells. 25 uls of serum matrix was added to the standards, controls and background, 25 ul of plasma was added to the sample wells followed by 25 ul of beads. This was incubated overnight on a shaking plate at 4° C. Fluid was removed gently by vacuum and the plates washed 2 times with 200 uls of wash buffer. 25 ul of detection antibody was added and incubated with shaking for 1 hour at room temperature. 25 ul streptavidin-Phycoerythrin was added and incubated 30 min with shaking. Fluid was removed gently by vacuum and washed 3 times. 100 ul sheath fluid was added and the beads resuspended on a shaker plate for 5 min. The plate was then run on the Luminex 100 IS according to manufacturer's directions. Samples were run in duplicate.

36 samples (n=12) were run for young (<5 years of age), middle-aged (5-10 years of age) and old (>10 years of age).

Outlier detection: A resistant z-score rule is applied to the outlier detection algorithm.

$${z}_{i} = \frac{Xi - \overline{X}}{\overline{S}}$$

Where X̅ and S̅ are the median and MAD. An outlier is called if |zᵢ|>4. Outliers were excluded from further statistical treatments. Means and standard errors were calculated. Results are shown in Table 4.

TABLE 4

| | Young | Middle | Old |
|---|---|---|---|
| Protein | Mean ± SE | Mean ± SE | Mean ± SE |
| GMCSF | 78.78 ± 18.27 | 27.76 ± 6.97 | 34.67 ± 5.74 |
| IFN | 8.54 ± 1.77 | 16.91 ± 3.14 | 12.66 ± 4.4 |
| IL-10 | 8.21 ± 1.88 | 8.48 ± 2.35 | 7.5 ± 1.31 |
| IL-15 | 77.32 ± 20.39 | 40.83 ± 8.74 | 78.95 ± 49.36 |
| lL-18 | 57.4 ± 18.18 | 22.69 ± 6.9 | 20.82 ± 3.11 |
| IL-2 | 17.45 ± 5.58 | 4.79 ± 1.98 | 26.55 ± 13.66 |
| IL-4 | 296.64 ± 106.58 | 120 ± 10.92 | 367.22 ± 96.75 |
| IL-7 | 17.71 ± 3.19 | 12.06 ± 1.86 | 11.37 ± 1.75 |
| IL-8 | 907.02 ± 133.16 | 651.3 ± 94.1 | 1178.48 ± 201.85 |
| IP-10 | 2.97 ± 0.09 | 2.88 ± 0.18 | 2.82 ± 0.08 |
| KC | 2082.88 ± 234.62 | 1039.3 ± 210.45 | 1478.58 ± 198.49 |
| MCP-1 | 64.11 ± 6.9 | 41.16 ± 5.44 | 64.26 ± 14.88 |
| Adiponectin | 2.08E+07 ± 2.81E+06 | 1.15E+07 ± 1.19E+06 | 1_{:}11E+07 ± 1.56E+06 |

Statistical analysis. ANOVA: a two-way ANOVA analysis was performed to evaluate the effects of the two factors: age (young, intermediate, old,) and gender (M, F) as well as their interaction. P values for both factors and their interaction are computed (p<0.05). Fitted value and standard error for each age group are also reported.

T-test: pair-wise T-test was used to compare the difference between means of the three age groups. Multiple comparisons are adjusted using Hommel's method to control family-wise error. P values are computed (p<0.05). Results are shown in Table 5.

TABLE 5

| Protein | P Age | P Yng-Mid | P Yng-Old | P Mid-Old |
|---|---|---|---|---|
| GMCSF | 0.0107 | 0.0164 | 0.0292 | 0.6858 |
| IFN | 0.4917 | 0.5201 | 0.5201 | 0.5201 |
| IL-10 | 0.9486 | 0.931 | 0.931 | 0.931 |
| IL-15 | 0.6396 | 0.9291 | 0.9733 | 0.8444 |
| IL-18 | 0.0508 | 0.0721 | 0.0625 | 0.9061 |
| IL-2 | 0.3034 | 0.4937 | 0.4937 | 0.3845 |
| IL-4 | 0.4411 | 0.6092 | 0.7159 | 0.4569 |
| IL-7 | 0.1175 | 0.2026 | 0.152 | 0.8365 |
| IL-8 | 0.1068 | 0.2751 | 0.2751 | 0.0895 |
| IP-10 | 0.7826 | 0.7939 | 0.7939 | 0.7939 |
| KC | 0.01 | 0.0053 | 0.1218 | 0.1489 |
| MCP-1 | 0.2219 | 0.2783 | 0.9922 | 0.2563 |
| Adiponectin | 0.0019 | 0.0028 | 0.0024 | 0.8861 |

The specification has disclosed typical preferred embodiments of the invention. Although specific terms are employed, they are used in a generic and descriptive sense only and not for purposes of limitation, the scope of the invention being set forth in the claims. Clearly, many modifications and variations of the invention are possible in light of the above teachings. It is therefore to be understood that within the scope of the appended claims the invention may be practiced otherwise than as specifically described.

What is claimed is:

1. A combination comprising a plurality of biomarkers associated with immune function that are differentially expressed in samples from old animals compared with samples from young animals, wherein the biomarker associated with immune function is one or more gene expression marker selected from E2F4, ADORA2A, RBMX, MVP, PEA15, UTP3, BST2, SORBS3, CD74, CD24, CCND3, PRKAG2, MED15, DNAJC8, CNDP2, CFD, IFNGR2, GABPA, TLR8, CAPG, GOT2, ZYX, MOV10, VDAC3, GNB2L1, NCF4, RPL7, SETD1B, NUDCD3, CD151, UIMC1, PADI4, TMEM55B, UPP1, GLTSCR2, MBOAT1, C22orf36, HSPB6, MSH2, ZNFX1, KDELR1, TMED10, SREBF1, IQGAP1, GPR177, HSPA6, TBCB, TRUB2, SUV39H1, GABARAP, PRKCSH, CD9, ZNF598, GPI, NUDC, TBC1D1, ADC, GAPDH, MED8, PSMC4, ATXN7L3, NCF1, GLIPR2, PEX19, MINPP1, PTPN23, PKM2, FLJ20160, FCGR1B, ADPGK, CIAPIN1, ARHGDIA, RPAP1, CCDC61, SYVN1, PADI4, DDOST, TREX1, PDCD11, TTC31, MAP7D1, MAPKSP1, HPX, DDOST, DERL2, TGFB1, PIM1, MAN2B1, USP3, RNH1, EIF4B, RHOG, SLC25A1, ACSS2, DOK2, NUMB, UCP2, VDAC3, LOC401875, ANXA11, PHKG2, GLB1, NARS, CLK3, AGBL5, PPP2R5C, XPNPEP1, TUBA4A, JARID1C, ARL4C, G6PC3, FES, USP5, and IREB2.

2. The combination of claim 1 wherein the biomarker associated with immune function is one or more gene expression marker selected from E2F4, ADORA2A, RBMX, MVP, PEA15, UTP3, BST2, SORBS3, CD74, CD24, CCND3, PRKAG2, MED15, DNAJC8, CNDP2, CFD, IFNGR2, GABPA, TLR8, CAPG, GOT2, ZYX, MOV10, VDAC3, GNB2L1, NCF4, RPL7, SETD1B, NUDCD3, CD151, and UIMC1.

3. A combination comprising a plurality of biomarkers associated with immune function that are differentially expressed in samples from old animals compared with samples from young animals, wherein the biomarker associated with immune function is one or more proteins selected from granulocyte-macrophage colony-stimulating factor (GMCSF), adiponectin, and interleukin-18 (IL-18).

4. The combination of claim 3 wherein the animal is a companion animal.

5. The combination of claim 4 wherein the companion animal is a canine.

6. A combination comprising a plurality of biomarkers associated with immune function that are differentially expressed in samples from middle-aged animals compared with samples from young animals, wherein the biomarker associated with immune function is one or more proteins selected from granulocyte-macrophage colony-stimulating factor (GMCSF), chemokine (C-X-C motif) ligand 1 (CXCL1), adiponectin, and interleukin-18 (IL-18).

7. The combination of claim 6 wherein the animal is a companion animal.

8. The combination of claim 7 wherein the companion animal is a canine.

9. A method for determining if a composition is effective in strengthening the immune function in an animal comprising:
a. obtaining a baseline sample from the animal prior to administration of the composition;
b. analyzing the baseline sample for one or more biomarkers associated with immune function;
c. administering the composition to the animal for a suitable amount of time;
d. obtaining a treatment sample from the animal after completion of the suitable amount of time;
e. analyzing the treatment sample for one or more biomarkers associated with immune function; and
f. determining if the composition is effective if one or more biomarkers present in the baseline sample is differentially expressed in the treatment sample.

10. The method of claim 9 wherein determining if the composition is effective if two or more biomarkers present in the baseline sample are differentially expressed in the treatment sample.

11. The method of claim 9 wherein determining if the composition is effective if three or more biomarkers present in the baseline sample are differentially expressed in the treatment sample.

12. The method of claim 9 wherein the biomarker associated, with immune function is one or more gene expression marker selected from E2F4, ADORA2A, RBMX, MVP, PEA15, UTP3, BST2, SORBS3, CD74, CD24, CCND3, PRKAG2, MED15, DNAJC8, CNDP2, CFD, IFNGR2, GABPA, TLR8, CAPG, GOT2, ZYX, MOV10, VDAC3, GNB2L1, NCF4, RPL7, SETD1B, NUDCD3, CD151, UIMC1, PADI4, TMEM55B, UPP1, GLTSCR2, MBOAT1, C22orf36, HSPB6, MSH2, ZNFX1, KDELR1, TMED10, SREBF1, IQGAP1, GPR177, HSPA6, TBCB, TRUB2, SUV39H1, GABARAP, PRKCSH, CD9, ZNF598, GPI, NUDC, TBC1D1, ADC, GAPDH, MED8, PSMC4, ATXN7L3, NCF1, GLIPR2, PEX19, MINPP1, PTPN23, PKM2, FLJ20160, FCGR1B, ADPGK, CIAPIN1, ARHGDIA, RPAPI, CCDC61, SYVN1, PADI4, DDOST, TREX1, PDCD11, TTC31, MAP7D1, MAPKSP1, HPX, DDOST, DERL2, TGFB1, PIM1, MAN2B1, USP3, RNH1, EIF4B, RHOG, SLC25A1, ACSS2, DOK2, NUMB, UCP2, VDAC3, LOC401875, ANXA11, PHKG2, GLB1, NARS, CLK3, AGBL5, PPP2R5C, XPNPEP1, TUBA4A, JARID1C, ARL4C, G6PC3, FES, USP5, and IREB2.

13. The method of claim 9 wherein the biomarker associated with immune function is one or more gene expression marker selected from E2F4, ADORA2A, RBMX, MVP, PEA15, UTP3, BST2, SORBS3, CD74, CD24, CCND3, PRKAG2, MED15, DNAJC8, CNDP2, CFD, IFNGR2, GABPA, TLR8, CAPG, GOT2, ZYX, MOV10, VDAC3, GNB2L1, NCF4, RPL7, SETD1B, NUDCD3, CD151, and UIMC1.

14. The method of claim 9 wherein the animal is a companion animal.

15. The method of claim 14 wherein the companion animal is a canine.

16. A method for determining if an animal is responding to treatment with a composition suitable for strengthening immune function comprising:
a. obtaining a baseline sample from the animal prior to administration of the composition;
b. analyzing the baseline sample for one or more biomarkers associated with immune function;
c. administering the composition to the animal for a suitable amount of time;
d. obtaining a treatment sample from the animal after completion of the suitable amount of time;
e. analyzing the treatment sample for one or more biomarkers associated with immune function; and
f. determining if the animal is responding to treatment if one or more biomarker present in the baseline sample is differentially expressed in the treatment sample.

17. The method of claim 16 wherein determining if the animal is responding to treatment if two or more biomarkers present in the baseline sample are differentially expressed in the treatment sample.

18. The method of claim 16 wherein determining if the animal is responding to treatment if three or more biomarkers present in the baseline sample are differentially expressed in the treatment sample.

19. The method of claim 16 wherein the biomarker associated with immune function is one or more gene expression marker selected from E2F4, ADORA2A, RBMX, MVP, PEA15, UTP3, BST2, SORBS3, CD74, CD24, CCND3, PRKAG2, MED15, DNAJC8, CNDP2, CFD, IFNGR2, GABPA, TLR8, CAPG, GOT2, ZYX, MOV10, VDAC3, GNB2L1, NCF4, RPL7, SETD18, NUDCD3, CD151, UIMC1, PADI4, TMEM55B, UPP1, GLTSCR2, MBOAT1, C22orf36, HSPB6, MSH2, ZNFX3, KDELR1, TMED10, SREBF1, IQGAP1, GPR177, HSPA6, TBCB, TRUB2, SUV39H1, GABARAP, PRKCSH, CD9, ZNF598, GPI, NUDC, TBC1D1, ADC, GAPDH, MED8, PSMC4, ATXN7L3, NCF1, GLIPR2, PEX19, MINPP1, PTPN23, PKM2, FLJ20160, FCGR1B, ADPGK, CIAPIN1, ARHGDIA, RPAP1, CCDC61, SYVN1, PADI4, DDOST, TREX1, PDCD11, TTC31, MAP7D1, MAPKSP1, HPX, DDOST, DERL2, TGFB1, PIM1, MAN2B1, USP3, RNH1, EIF4B, RHOG, SLC25A1, ACSS2, DOK2, NUMB, UCP2, VDAC3, LOC401875, ANXA11, PHKG2, GLB1, NARS, CLK3, AGBL5, PPP2R5C, XPNPEP1, TUBA4A, JARID1C, ARL4C, G6PC3, FES, USP5, and IREB2.

20. The method of claim 16 wherein the biomarker associated with immune function is one or more gene expression marker selected from E2F4, ADORA2A, RBMX, MVP, PEA15, UTP3, BST2, SORBS3, CD74, CD24, CCND3, PRKAG2, MED15, DNAJC8, CNDP2, CFD, IFNGR2, GABPA, TLR8, CAPG, GOT2, ZYX, MOV10, VDAC3, GNB2L1, NCF4, RPL7, SETD1B, NUDCD3, CD151, and UIMC1.

21. The method of claim 16 wherein the animal is a companion animal,

22. The method of claim 21 wherein the companion animal is a canine.
